(19) United States
(12) Patent Application Publication
(10) Pub. No.: US 2013/0217779 A1
(43) Pub. Date: Aug. 22, 2013

(54) METHADONE CHIRAL ISOLATE AS AN IMPROVED PHARMACEUTICAL

(71) Applicants: John C. Somberg, Lake Bluff, IL (US); Vasant Ranade, Lake Bluff, IL (US)

(72) Inventors: John C. Somberg, Lake Bluff, IL (US); Vasant Ranade, Lake Bluff, IL (US)

(73) Assignee: ACADEMIC PHARMACEUTICALS INCORPORATED, Lake Bluff, IL (US)

(21) Appl. No.: 13/528,365

(22) Filed: Jun. 20, 2012

Related U.S. Application Data
(63) Continuation of application No. 12/454,373, filed on May 18, 2009, now abandoned.
Application No. 12/454,373 is a continuation of application No. 12/288,441, filed on Oct. 20, 2008, now abandoned.
Application No. 12/288,441 is a continuation-in-part of application No. 11/588,157, filed on Oct. 27, 2006, now abandoned.

(51) Int. Cl. A61K 31/137

(52) U.S. Cl. CPC A61K 31/137
USPC 514/648

(57) ABSTRACT

The present invention relates to a method for treating drug addiction or neuropathic pain in a patient known to be vulnerable to life threatening cardiac arrhythmias (e.g., abnormally prolonged QT intervals, potassium chanelopathies, hypokalemia, or a combination thereof) by administering the R isolate of methadone to the patient.

This application is a continuation in part of application Ser. No. 11/588,157 first filed on Oct. 27, 2006 and a continuation of the refilling of patent on Oct. 20, 2008 application Ser. No. 12/288,441.

### BACKGROUND OF THE INVENTION

Methadone is an important long-term maintenance therapy for drug addiction that is very frequently employed in clinical practice. Additionally, methadone is used as an analgesic therapy. It has been found to prolong the QT interval on the surface electrocardiogram, QT prolongation having been associated with the development of serious arrhythmias, especially of the Torsade de Pointes type of ventricular tachycardia. Methadone's prolongation of cardiac repolarization, which is recognized on the EKG as QT interval prolongation can facilitate arrhythmias. Thus, QT interval prolongation is associated with serious arrhythmias and has caused the withdrawal of drugs such as cissipride, a drug used to improve GI motility and terfenadine, a non-sedating antihistime. Methadone, being a chiral drug, can be separated into R&S form (FIG. 1) with each isolate possessing different pharmacologic properties. The isolation of a chiral methadone isolate without QT prolongation or with a reduced QT prolonging effect could increase the therapeutic index of methadone, making it a safer chronic therapy for abuse prone patients or a safer therapy for the acute treatment of pain. This is especially the case with a drug that is itself subject to patient administered overdosage, leading to excessive QT prolongation or repolarization heterogeneity that can develop into the cardiac arrhythmia, Torsade de Pointes ventricular tachycardia, a life threatening arrhythmia.

Methadone is primarily a μ agonist with pharmacologic properties similar to those of morphine. It is one of the analogs of pethidine developed by I.G. Farben Industries just before World War II by Max Bock Mohl & Gustav Ehrhart. The compound was named amiodone and in high doses was reported to possess serious side effects. A U.S. investigation of I.G. Farben after WWII found information on amiodon and provided this information to the Committee on Drug Addiction and Narcotics with the resulting report published by Eddy in 1947 (J. Am. Pharm. Assoc. Pract. Pharm. Ed. 1947, 8:336). The compound was officially named methadone and was found to be an alternative to morphine for pain relief, causing less sedation and less depression. However, the drug was still addictive, but with a reduced euphoric effect, making long acting methadone a candidate therapy for chronic treatment of drug addiction to agents such as heroin.

Methadone is also used for the treatment of neuropathic pain and its use in this area has recently undergone a resurgence. However, a limitation of methadone's use has been QT prolongation and the potential for life threatening arrhythmias. A methadone overdose may be associated with serious ventricular arrhythmias and sudden death. However, arrhythmias can occur at standard doses as well, making the administration of standard doses hazardous at times, especially in vulnerable patients. Given the abuse potential and the potential for overdosing on methadone, removing the arrhythmic potential would greatly improve the safety margin. However, even without taking an overdose or an excessively high dose, QT prolongation may pose a risk to female patients, patients who are hypokalemic or patients with underlying abnormalities with their potassium channels. Certain individuals can have an increased risk of fatal arrhythmias with QT prolonging drugs. Hypokalemia increases the potential for serious arrhythmias. Female gender increases risk, since women have longer QT intervals without any concomitant drug therapy. Patients with congenital long QT interval syndrome are at increased risk and in fact, recent observations have found many susceptible individuals with point mutations in the gene expressing the ion channel, making for an increased susceptibility to drug induced arrhythmias. Given the potential for lethal arrhythmias, removing or diminishing the QT prolonging effects would increase the safety of methadone, permitting higher doses to be employed without increasing the risk of serious life threatening arrhythmias.

### SUMMARY OF THE INVENTION

Described are two isolates of methadone and a method of chirally separating methadone into at least two isomers, one of the isolates having little or no effect on the IKr channel and consequently not causing QT prolongation or arrhythmias.

### BRIEF DESCRIPTION OF THE DRAWINGS

FIG. 1: Illustrates the chemical structure of methadone.

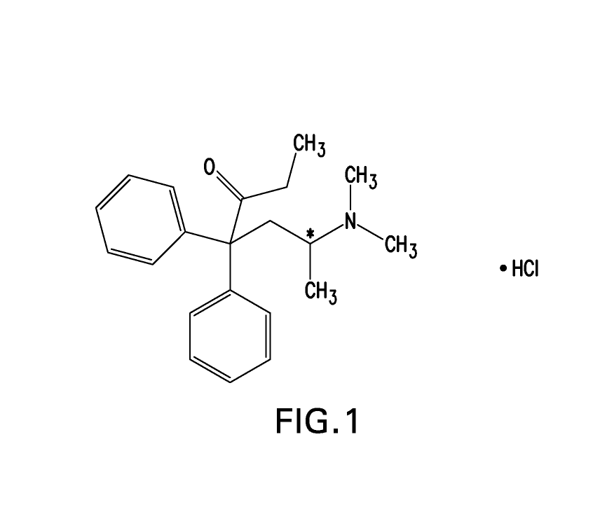

FIG. 2: HPLC chromatogram of the chiral separation of methadone.

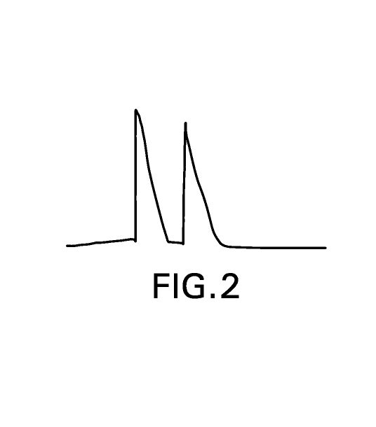

FIG. 3: A graph illustrating the percentage current block by increasing methadone concentration.

FIG. 4: The percentage of current blocked by incoming concentration of R&S isomers of methadone.

FIG. 5: The percentage of current blocked by increasing concentration of R&S isomers of methadone adjusted for analgesic potency.

### DETAILED DESCRIPTION OF THE INVENTION

To separate methadone, an HPLC system using a cyclobond column was employed. The chromatographic system consisted of a spectra-physics device employing a solvent (SP2000) with a chronjet injector. The column effluent was monitored by a Spectra-Physics variable UV wavelength detector set at 254 nm. The variable phase consisted of CH₃CN:H₂O (1-3 v/v), pH 7.1 and the effluent was pumped at a rate of 1 ml/min. Injecting 10 micro liters of 1 mg/ml material in ethanol resulted in obtaining two chromatographic peaks and with different retention times of 1.44 and 2.2. There was a 0.8 cm difference in the two retention times. The two isolates were separately collected and then concentrated, reconstituted and then tested using a polarimeter and mass spectroscopy. The optical rotations were found to be −143° and 138°. The mass spectroscopy showed the molecular weight of methadone to be 310 and this was the same for the two isolates. The mass is 310 not 345, since the HCl is not included in the spectrographic measurement.

### Determination of Optical Rotation:

Optical rotation of methadone isolates (peak 1 and peak 2) were determined using a Perkin-Elmer instrument. Approximately 0.5 mg/ml concentration of the isolates was employed and the optical readings were recorded. The material was suspended in 1.5 mg/ml solution of alcohol. Alcohol blanks showed no optical activity. The optical rotation for peak 1 was −143° and the optical activity for peak 2 was +138° The optical rotation of methadone racemate was 0°.

### Mass Spectroscopy Analysis:

Mass spectroscopy analysis was obtained using a Micro Mass UK mass spectrometer. The mobile phase consisted of CH₃CN:H₂O (1:3 v/v), pH 7.1 and the flow rate was 0.4 ml/min. Waters 996 PDA was used as a UV wavelength detector with a starting wavelength at 254 nm and a bandwidth of 500 nm. The results showed that the racemate and the two isomers had the same molecular weight.

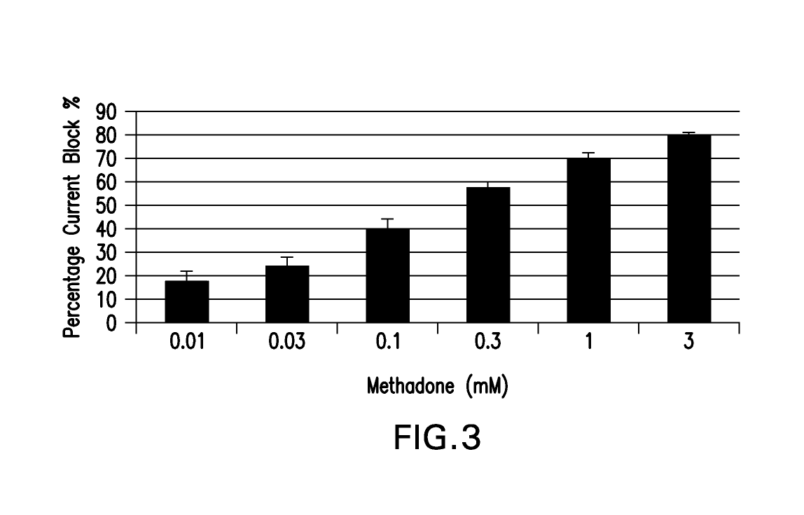

To detect an effect of a pharmacologic agent on the QT interval, a model of IKr inhibition was used. Not all agents that prolong the QT interval are readably detected in an in vitro system. Thus studies were undertaken with the methadone racemate to determine the effect on IKr inhibition in xenopus oocytes. In this model DNA is cloned, linerarlized to cRNA and injected into xenopus oocytes to overexpress the IKr channel. Two electrode voltage clamp techniques are then employed to make recordings from the oocytes exposed to solvent recording solution, and incremental concentrations of methadone are added with electrophysiologic recordings made of current flow across the membrane. The results of studying concentrations of methadone 0.01 to 3 mM are presented in FIG. 3. The figure shows a dose related inhibition of the IKr current by methadone.

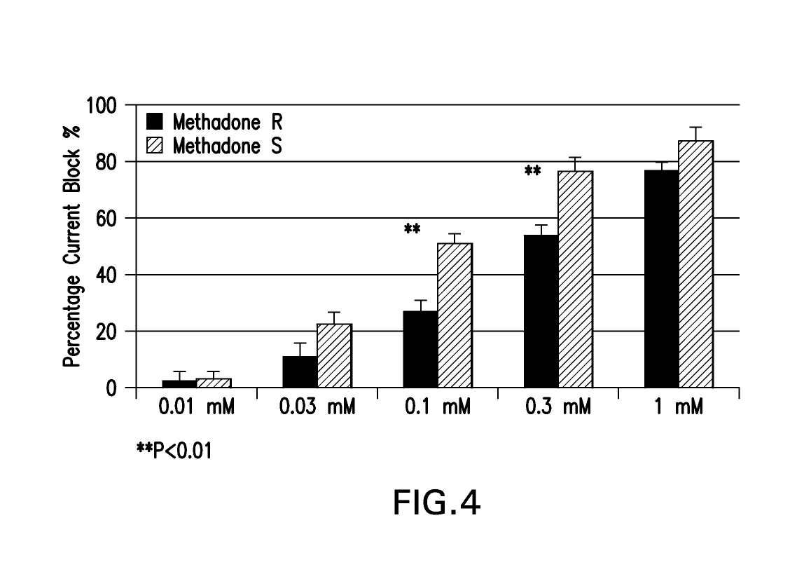

Additionally, the two chiral isolates were evaluated as to inhibition of IKr, 0.1 mM to 1 mM (FIG. 4). S-methadone showed a greater block in potassium current at each dose across the concentration range.

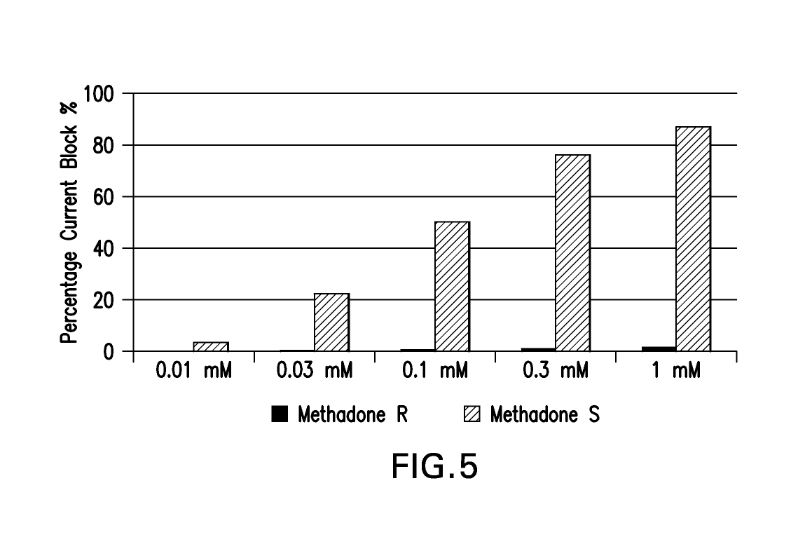

The S isomer has one fiftieth the analgesic potency of the R isomer of methadone (Scott C C, Robbins E B, Chen K K. Pharmacological comparison of the optical isomers of methadone. J Pharmacol Exp Ther. 1948; 93(3):282-6). When one adjusts the dose of R methadone to be 1/50 that of S methadone, the correct dose ratio for equipotent analgesic action, one then can compare the percentage current block in potassium IKr channel as expressed in oocytes. As seen in FIG. 5, S methadone significantly inhibits the potassium current to a greater degree than R methadone across a very broad concentration range (0.01 mM to 1 mM).

1. (canceled)

2. (canceled)

3. (canceled)

4. (canceled)

5. (canceled)

6. A method for treating drug addiction in a patient known to be vulnerable to life threatening cardiac arrhythmias, comprising: administering the R isolate of methadone to the patient.

7. The method of claim 6, wherein the patient is female or is known to have abnormally prolonged QT intervals, potassium chanelopathies, hypokalemia, or a combination thereof.

8. The method of claim 6, wherein the patient is female.

9. The method of claim 6, wherein the patient is known to have long QT intervals.

10. The method of claim 6, wherein the patient is known to have potassium chanelopathies.

11. The method of claim 6, wherein the patient is known to have hypokalemia.

12. The method of claim 6, wherein R isolate of methadone is substantially free of the S isolate.

13. The method of claim 6, wherein the R isolate of methadone is administered topically, rectally, orally, or intravenously.

14. The method of claim 13, wherein the R isolate of methadone is administered intravenously.

15. The method of claim 6, wherein the R isolate of methadone is administered in a sustained delivery system to prolong action in the body.

16. The method of claim 15, wherein the R isolate of methadone is administered intravenously.

17. A method a treating neuropathic pain in a patient known to be vulnerable to life threatening cardiac arrhythmias, comprising: administering the R isolate of methadone to the patient.

18. The method of claim 17, wherein the patient is female or is known to have abnormally prolonged QT intervals, potassium chanelopathies, hypokalemia, or a combination thereof.

19. The method of claim 17, wherein the patient is female.

20. The method of claim 17, wherein the patient is known to have long QT intervals.

21. The method of claim 17, wherein the patient is known to have potassium chanelopathies.

22. The method of claim 17, wherein the patient is known to have hypokalemia.

23. The method of claim 17, wherein R isolate of methadone is substantially free of the S isolate.

24. The method of claim 17, wherein the R isolate of methadone is administered topically, rectally, orally, or intravenously.

25. The method of claim 24, wherein the R isolate of methadone is administered intravenously.

26. The method of claim 17, wherein the R isolate of methadone is administered in a sustained delivery system to prolong action in the body.

27. The method of claim 26, wherein the R isolate of methadone is administered intravenously.
